(19) United States
(12) Patent Application Publication
(10) Pub. No.: US 2023/0363808 A1
(43) Pub. Date: Nov. 16, 2023

(54) Orthopedic Screw with Radiolucent and Radiopaque Thread Portions

(71) Applicant: GLW, Inc., Englewood Cliffs, NJ (US)

(72) Inventors: Santiago Suniaga Da Silva, Hamburg (DE); Axel Cremer, Fahrenkrug (DE)

(73) Assignee: GLW, Inc., Englewood Cliffs, NJ (US)

(21) Appl. No.: 18/196,210

(22) Filed: May 11, 2023

Related U.S. Application Data
(60) Provisional application No. 63/341,192, filed on May 12, 2022.

(51) Int. Cl. A61B 17/86

(52) U.S. Cl. CPC A61B 17/866; A61B 17/8625

(57) ABSTRACT

The disclosure relates to orthopedic screws and methods of manufacturing orthopedic screws. An orthopedic screw has an inner core member defining a proximal radiopaque thread and a distal radiopaque thread, and an outer body member defining a radiolucent outer body member external thread that is continuous with the proximal radiopaque thread and the distal radiopaque thread.

### FIELD

The disclosure relates to the field of medical devices. More particularly, the disclosure relates to implantable medical devices useful for orthopedic applications. Specific examples relate to orthopedic screws. The disclosure also relates to methods of manufacturing a medical device, including methods of manufacturing an orthopedic screw.

### BACKGROUND

Orthopedic screws, also referred to as bone screws, are implantabled medical devices that are commonly used for fracture stabilization and fixation. These devices can be made from a variety of materials and can include structural adaptations that facilitate their use, enhance their performance, or both. For example, some orthopedic screws define a cannula to enable placement over a wire.

While hybrid orthopedic screws are known, the inclusion of multiple materials in the construction of an orthopedic screw can result in handling and performance drawbacks.

A need remains, therefore, for improved orthopedic screws and methods of manufacturing an orthopedic screw.

### BRIEF SUMMARY OF SELECTED EXAMPLES

Various example orthopedic screws are described.

An example orthopedic screw comprises an inner core member defining first and second radiopaque threads, and an outer body member defining a radiolucent outer body member external thread that is continuous with the first and second radiopaque threads.

Another example orthopedic screw comprises an inner core member defining a proximal radiopaque thread and a distal radiopaque thread, and an outer body member defining a radiolucent outer body member external thread that is continuous with the proximal radiopaque thread and the distal radiopaque thread.

Another example orthopedic screw comprises an inner core member defining a proximal radiopaque thread, a distal radiopaque thread, and an intermediate radiopaque thread disposed between and axially spaced from the proximal radiopaque thread and the distal radiopaque thread, a first outer body member defining a first radiolucent outer body member external thread that is continuous with the proximal radiopaque thread and the intermediate radiopaque thread, and a second outer body member defining a second radiolucent outer body member external thread that is continuous with the intermediate radiopaque thread and the distal radiopaque thread.

Another example orthopedic screw comprises an inner core member having a head having a first outer diameter, a tip having a second outer diameter, and defining a circumferential recess having a third outer diameter that is less than the first outer diameter and the second outer diameter. The inner core member defines a proximal radiopaque thread and a distal radiopaque thread. An outer body member is disposed in the circumferential recess of the inner core member and defines a radiolucent outer body member external thread that is continuous with the proximal radiopaque thread and the distal radiopaque thread.

Another example orthopedic screw comprises an inner core member having a head having a first outer diameter, a tip having a second outer diameter, and a body extending between the head and the tip and defining a circumferential recess having a third outer diameter that is less than the first outer diameter and the second outer diameter. The inner core member defines a proximal radiopaque thread and a distal radiopaque thread. An outer body member is disposed in the circumferential recess of the inner core member and defines a radiolucent outer body member external thread that is continuous with the proximal radiopaque thread and the distal radiopaque thread.

Various example methods of manufacturing an orthopedic screw are described.

An example method of manufacturing an orthopedic screw comprises machining an inner core member from a precursor; placing the inner core member into a mold, and overmolding the inner core member with a material to form an outer body member about the inner core member to produce an orthopedic screw according to an embodiment.

Another example method of manufacturing an orthopedic screw comprises forming an inner core member from a suitable precursor, such as a solid rod or cannula such that the inner core member defines a proximal radiopaque thread, a distal radiopaque thread, and a circumferential recess; placing the inner core member into a suitable mold; and overmolding the inner core member with a suitable material to form an outer body member about the inner core member such that the outer body member is disposed in the circumferential recess of the inner core member and defines a radiolucent thread that is continuous with both the proximal radiopaque thread and the distal radiopaque thread.

Additional understanding of the claimed orthopedic screws and methods of manufacturing an orthopedic screw can be obtained by reviewing the detailed description of selected examples, below, with reference to the appended drawings.

### DESCRIPTION OF FIGURES

FIG. 1 is a side view of an example orthopedic screw.

FIG. 2 is a sectional view of the orthopedic screw illustrated in FIG. 1, taken along line 2-2.

FIG. 3 is a magnified view, partially broken away, of the distal portion of the orthopedic screw illustrated in FIG. 1.

FIG. 4 is sectional view, taken along line 4-4 in FIG. 3, of the distal portion of the orthopedic screw illustrated in FIG. 1.

FIG. 5 is a partial sectional view of the distal portion of the orthopedic screw illustrated in FIG. 1.

FIG. 6 is a partial sectional view of the distal portion of an alternative orthopedic screw.

FIG. 7 is a partial sectional view of the distal portion of another alternative orthopedic screw.

FIG. 8 is a partial sectional view of the distal portion of another alternative orthopedic screw.

FIG. 9 is a side view of another example orthopedic screw.

FIG. 10 is a sectional view of the orthopedic screw illustrated in FIG. 9, taken along line 10-10.

FIG. 11 is a partial sectional view of the distal portion of the orthopedic screw illustrated in FIG. 9.

FIG. 12 is a partial sectional view of the orthopedic screw illustrated in FIG. 9.

FIG. 13 is a partial sectional view of an alternative orthopedic screw.

FIG. 14 is a partial sectional view of another alternative orthopedic screw.

FIG. 15 is a flowchart representation of an example method of manufacturing an orthopedic screw.

### DETAILED DESCRIPTION OF SELECTED EXAMPLES

The following detailed description and the appended drawings describe and illustrate various example orthopedic screws and methods of manufacturing an orthopedic screw. The description and illustration of these examples enable one skilled in the art to make and use examples of the inventive orthopedic screws and to perform examples of the inventive methods of manufacturing an orthopedic screw. They do not limit the scope of the claims in any manner.

As used herein, the term “lumen,” and grammatically related terms, refers to the inside space of a tubular structure. The term does not require any specific dimensions, relative dimensions, configuration, or regularity.

As used herein, the term “circumferential,” and grammatically related terms, refers to a structural arrangement of one structure relative to another structure, feature, or property of another structure. The term does not require any specific dimensions, relative dimensions, configuration, or regularity of either structure.

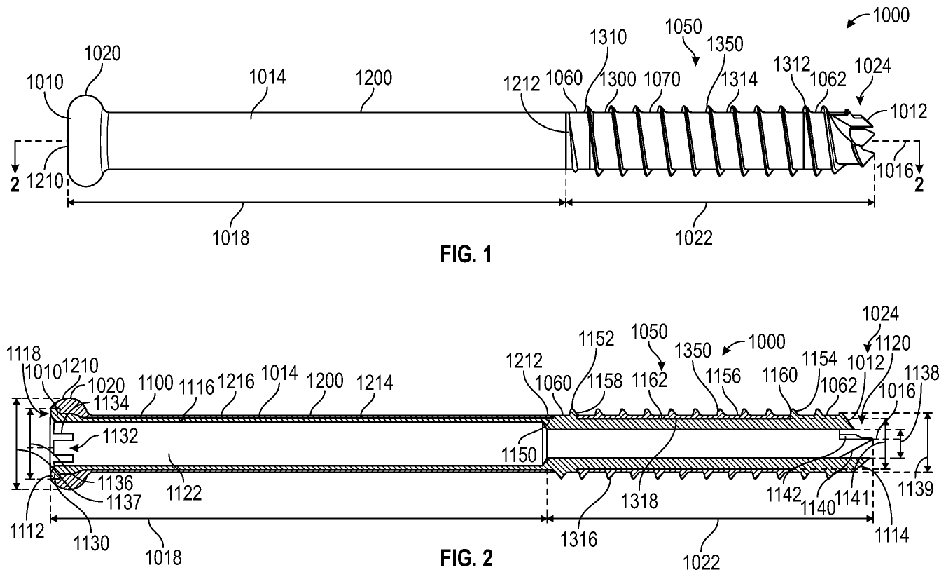

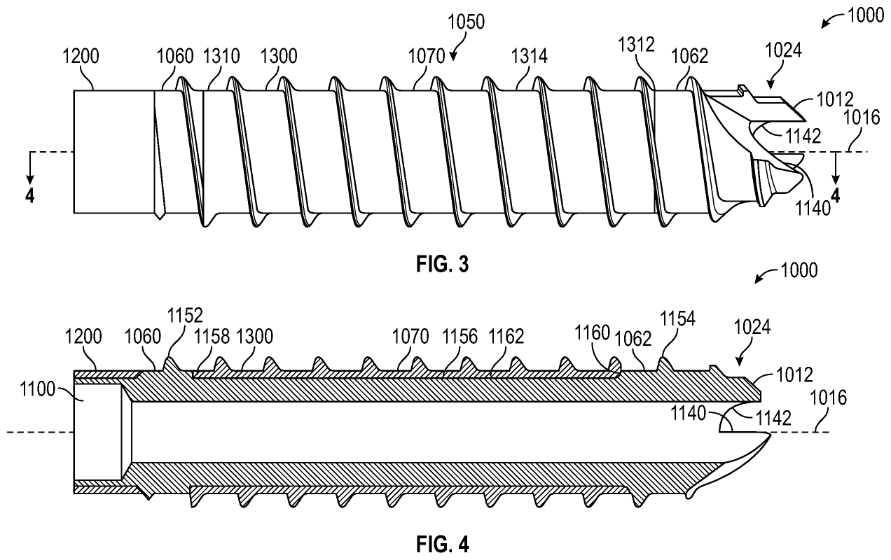

Each of FIGS. 1, 2, 3, and 4 illustrates a first example orthopedic screw 1000 or a portion of the orthopedic screw 1000. The orthopedic screw 1000 has a proximal end 1010, a distal end 1012, and a body 1014 extending between the proximal end 1010 and the distal end 1012 along a longitudinal axis 1016. A proximal axial portion 1018 extends from the proximal end 1010 toward, but not to, the distal end 1012 and includes a head 1020. A distal axial portion 1022 extends from the distal end 1012 toward, but not to, the proximal end 1010 and includes a distal tip 1024. As described in detail below, the head 1020 defines a cavity for receving an end of a tool for placing or implanting the orthopedic screw 1000 into tissue, such as a driver. Also as described in detail below, the distal tip 1024 is a cutting tip that defines structure for cutting into tissue, such as bone, cartilage, or both, as the orthopedic screw is driven by a tool. As described in detail below, orthopedic screw 1000 includes a thread 1050 that includes a proximal radiopaque portion 1060, a distal radiopaque portion 1062, and a radiolucent portion 1070 extending between the proximal radiopaque portion 1060 and the distal radiopaque portion 1062.

The orthopedic screw 1000 includes an inner core member 1100, a proximal outer body member 1200 disposed circumferentially around a proximal portion of the inner core member 1100, and a distal outer body member 1300 disposed circumferentially around a distal portion of the inner core member 1100. Also as described in detail below, the orthopedic screw 1000 is a cannulated screw, allowing it to be passed over a separate member, such as a wire, to facilitate placement and/or positioning during implantation.

The inner core member 1100 has a proximal end 1112, a distal end 1114, and a body 1116 extending between the proximal end 1112 and the distal end 1114 along the longitudinal axis 1016 of the orthopedic screw 1000. The proximal end 1112 defines a proximal opening 1118 and the distal end 1114 defines a distal opening 1120. As best illustrated in FIG. 2, the inner core member 1100 defines a lumen 1122 extending between the proximal opening 1118 and the distal opening 1120. Thus, each of the proximal opening 1118 and the distal opening 1120 provides access to the lumen 1122 from the environment external to the orthopedic screw 1000. In use, the orthopedic screw 1000 can be passed over a previously-placed wire such that the wire extends through the lumen 1122. The orthopedic screw 1000 can then be advanced over such a wire to achieve a desired placement and/or positioning before initiating driving of the orthopedic screw 1000 into tissue.

The inner core member 1100 defines a head 1130 at the proximal end 1112 and a tip 1140 at the distal end 1114. The head 1130 defines a cavity 1132 that is bounded by a circumferential wall 1134. The circumferential wall 1134 surrounds the longitudinal axis 1016 of the orthopedic screw 1000. The inner core member 1100 transitions from a larger inner diameter 1136, present in the head 1130, to a smaller inner diameter 1138, present at the tip 1140.

The circumferential wall 1134 defines structure that facilitates interaction with a tool, such as a driver, that can be used to implant drive the orthopedic screw 1000 into tissue, such as cartilage and/or bone. As such, the circumferential wall 1134, and, as a result, the cavity 1132 may have any suitable configuration and a skilled artisan will be able to select an appropriate configuration for each of these structures in an orthopedic screw according to a particular embodiment based on various considerations, including the configuration and nature of any driver with which the orthopedic screw is intended to be used. Examples of suitable configurations include conventional configurations for screw heads, including hex-shaped configurations, star-shaped configurations, such as configurations compatible with TORX brand drivers, and other configurations. In this example, the head 1130 is an enlarged structure relative to the tip 1140 and body 1116 of the inner core member 1100.

The tip 1140 is positioned at the distal end 1114 of the inner core member 1100 and defines cutting edge 1142. Cutting edge 1142 defines structure for cutting into tissue, such as bone, cartilage, or both. Cutting edge 1142 may have any suitable configuration and a skilled artisan will be able to select an appropriate configuration for the cutting edge in an orthopedic screw according to a particular embodiment based on various considerations, including the nature of any particular tissue with which the orthopedic screw is intended to be used. Examples of suitable configurations include conventional configurations for bone screw cutting edges. Different configurations may be appropriate for orthopedic screws intended for use in different tissues and/or applications. For example, an orthopedic screw intended for use in fracture fixation in the foot and/or ankle may have one cutting edge configuration while an orthopedic screw intended for us in fracture fixation in the upper extremities may have another, different cutting edge configuration. Orthopedic screws intended for other uses, such as spinal or trauma applications, may have yet other cutting edge configurations.

The body 1116 extends between the head 1130 and the tip 1140 along the longitudinal axis 1016 of the orthopedic screw 1000. As best illustrated in FIG. 2, the body 1116 has an outer surface 1150 that defines proximal thread 1152, distal thread 1154, and a circumferential recess 1156 disposed between proximal thread 1152 and distal thread 1154. Considering the nature of the material of the inner core member 1100, as described in detail below, proximal thread 1152 provides the proximal radiopaque portion 1060 of the thread 1050 and distal thread 1154 provides the distal radiopaque portion 1062 of the thread 1050.

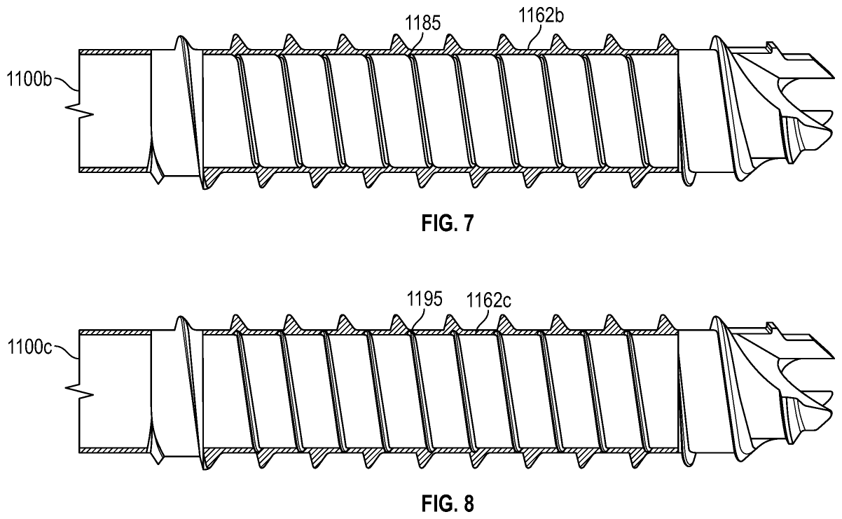

The circumferential recess 1156 extends entirely around the longitudinal axis 1008 of the orthopedic screw 1000. As best illustrated in FIGS. 2 and 4, the body 1116 defines a proximal recess wall 1158, a distal recess wall 1160, and a recess floor 1162 to form the circumferential recess 1156. As such, the circumferential recess 1156 is bounded by the proximal recess wall 1158, distal recess wall 1160, and recess floor 1162. As best illustrated in FIGS. 2 and 4, recess floor 1162 can be a substantially flat surface. Alternatively, recess floor 1162 can define structure that provides a larger surface area relative to the surface area provided by a flat or substantially flat surface. Such alternative recess floors are considered advantageous at least because they provide additional surface area for contact with an outer body member, as described below, which can increase the overall structural integrity of the orthopedic screw 1000. For example, FIG. 6 illustrates an inner core member 1100a having a recess floor 1162a that defines a plurality of circumferential grooves 1175. Each groove of the plurality of circumferential grooves 1175 is perpendicular to the longitudinal axis 1016 of the orthopedic screw 1000 and is distinct from all other grooves of the plurality of circumferential grooves 1175. In another example, illustrated in FIG. 7, an inner core member 1100b has a recess floor 1162b that defines a spiral groove 1185 that extends around the longitudinal axis 1016 of the orthopedic screw 1000. The spiral groove 1185 is continuous and can be described as a negative or inverse thread. In another example, illustrated in FIG. 8, an inner core member 1100c has a recess floor 1162c that defines a spiral projection 1195 that extends around the longitudinal axis 1016 of the orthopedic screw 1000. The spiral projection 1195 is continuous and can be described as a positive thread.

As best illustrated in FIG. 2, the inner core member 1100 has a first outer diameter 1137 at the head 1130, a second outer diameter 1139 at the tip 1140, and a third outer diameter 1141 at the circumferential recess 1156 of the body 1116. The first outer diameter 1137 is greater than both the second outer diameter 1139 and the third outer diameter 1141. Also, the second outer diameter 1139 is greater than the third outer diameter 1141.

Proximal outer body member 1200 has a proximal end 1210, a distal end 1212, and a body 1214 extending between the proximal end 1210 and the distal end 1212. As best illustrated in FIG. 2, the proximal outer body member 1200 defines a lumen 1216 extending between the proximal end 1210 and the distal end 1212. The body 1106 of the inner core member 1100 is disposed within the lumen 1216 of the proximal outer body member 1200. Also, the proximal end 1210 of the proximal outer body member 1200 extends over the head 1130 of the inner core member 1100. The distal end 1212 of the proximal outer body member 1200 is in contact with the portion of the inner core member 1100 that defines proximal thread 1152, which provides the proximal radiopaque portion 1060 of thread 1050 of the orthopedic screw 1000.

Distal outer body member 1300 is disposed in circumferential recess 1156 and circumferentially around inner core member 1100. Distal outer body 1300 extends between proximal thread 1152 and distal thread 1154 of the inner core member 1100, and defines an external thread 1350 that is continuous with both the proximal thread 1152 and distal thread 1154 of the inner core member 1100. Considering the nature of the material of the distal outer body member 1300, as described in detail below, external thread 1350 provides the radiolucent portion 1070 of the thread 1050. In this example, external thread 1350 extends along the entire axial length of the distal outer body member 1300.

Distal outer body member 1300 has a proximal end 1310, a distal end 1312, and a body 1314 extending between the proximal end 1310 and the distal end 1312. The distal outer body member 1300 defines a lumen 1316 extending between the proximal end 1310 and the distal end 1312. The body 1116 of the inner core member 1100 is disposed within and extends through the lumen 1316 of the distal outer body member 1300. The distal outer body member 1300 has an internal surface 1318 that is in contact interface with the recess floor 1162 of the inner core member 1100.

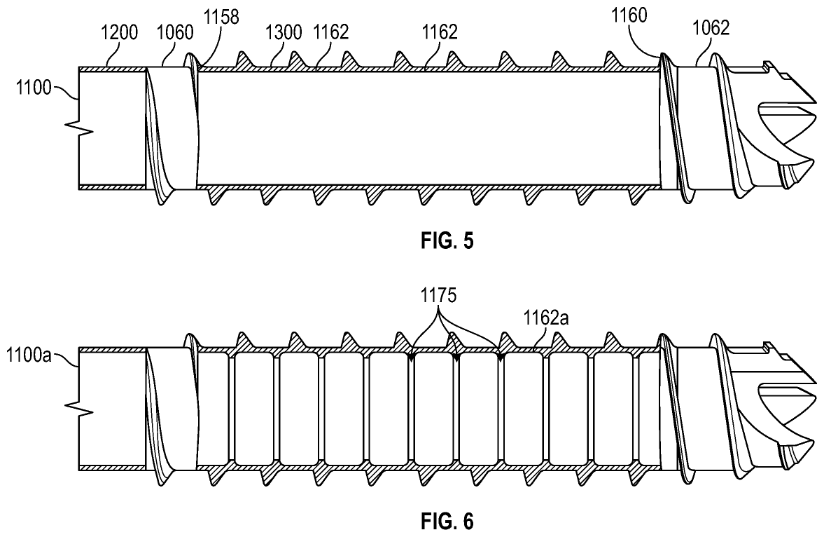

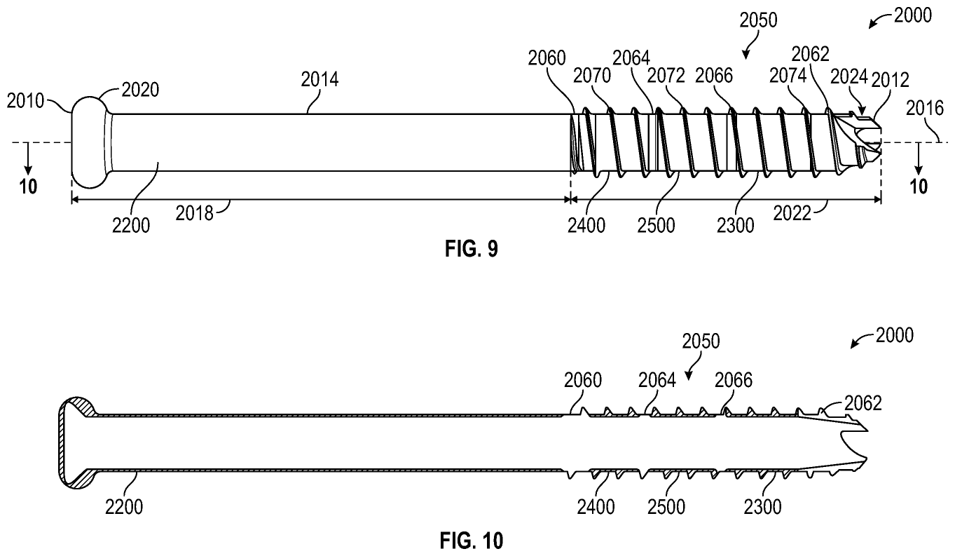

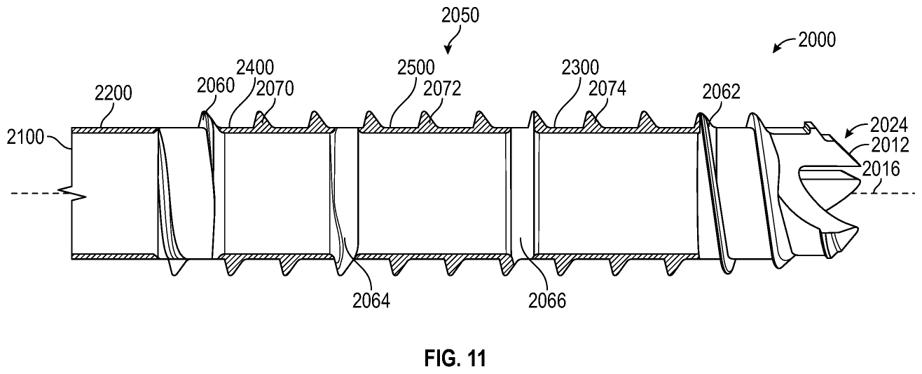

Each of FIGS. 9, 10, and 11 illustrates a second example orthopedic screw 2000 or a portion of the orthopedic screw 2000. Orthopedic screw 2000 is similar to orthopedic screw 1000 described above and illustrated in FIGS. 1, 2, 3, 4, and 5, except as detailed below. Thus, orthopedic screw 2000 has a proximal end 2010, a distal end 2012, and a body 2014 extending between the proximal end 2010 and the distal end 2012 along a longitudinal axis 2016. A proximal axial portion 2018 extends from the proximal end 2010 toward, but not to, the distal end 2012 and includes a head 2020. A distal axial portion 2022 extends from the distal end 2012 toward, but not to, the proximal end 2010 and includes a distal tip 2024. Head 2020 defines a cavity for receving an end of a tool for placing or implanting the orthopedic screw 2000 into tissue, such as a driver. Distal tip 2024 is a cutting tip that defines structure for cutting into tissue, such as bone, cartilage, or both, as the orthopedic screw is driven by a tool. Orthopedic screw 2000 includes a thread 2050 that includes a proximal radiopaque portion 2060 and a distal radiopaque portion 2062. In this example, thread 2050 also includes first 2064 and second 2066 intermediate radiopaque portions disposed between and separate from proximal 2060 and distal 2062 portions. Also in this example, thread 2050 includes and a first radiolucent portion 2070 extending between the proximal radiopaque portion 2060 and the first intermediate radiopaque portion 2064, a second radiolucent portion 2072 extending between the first intermediate radiopaque portion 2064 and the second intermediate radiopaque portion 2066, and a third radiolucent portion 2074 extending between the second intermediate radiopaque portion 2066 and the distal radiopaque portion 2062.

The orthopedic screw 2000 includes an inner core member 2100, a proximal outer body member 2200 disposed circumferentially around a proximal portion of the inner core member 2100, a distal outer body member 2300 disposed circumferentially around a distal portion of the inner core member 2100, a first intermediate outer body member 2400 disposed circumferentially around a first intermediate portion of the inner core member 2100 and axially between the proximal outer body member 2200 and the distal outer body member 2300, and a second intermediate outer body member 2500 disposed circumferentially around a second intermediate portion of the inner core member 2100 and axially between the proximal outer body member 2200 and the distal outer body member 2300. The orthopedic screw 2000 is a cannulated screw, allowing it to be passed over a separate member, such as a wire, to facilitate placement and/or positioning during implantation.

Inner core member 2100 defines the proximal radiopaque portion 2060, the first intermediate radiopaque portion 2064, the second intermediate radiopaque portion 2066, and the distal radiopaque portion 2062 of the thread 2050. The first intermediate outer body member 2400 defines the first radiolucent portion 2070 of the thread 2050. The second intermediate outer body member 2500 defines the second radiolucent portion 2072 of the thread 2050. The distal outer body member 2300 defines the third radiolucent portion 2074 of the thread 2050.

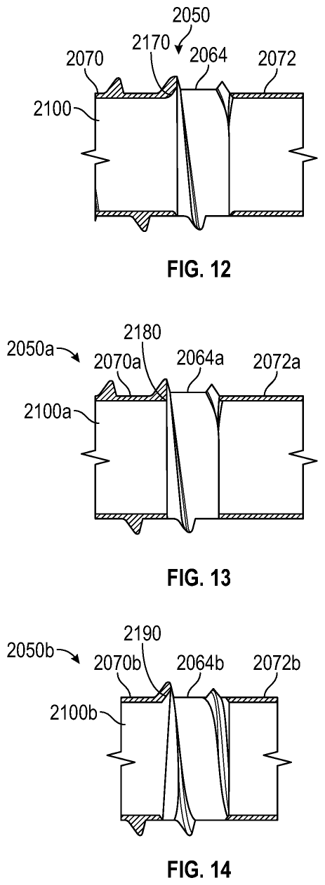

In the illustrated example, inner core member 2100 defines a radius 2170 at each transition from a portion of the inner core member 2100 that is disposed within one of the outer body members 2200, 2300, 2400, 2500 to a portion that defines a radiopaque thread 2060, 2062, 2064, 2066. Thus, as best illustrated in FIG. 12, inner core member defines radius 2170 at the transition from the portion of the inner core member 2100 that is disposed within first intermediate outer body member 2070 to the portion of the inner core member 2100 that defines first intermediate radiopaque portion 2064 of thread 2050. Other transition geometries are contemplated. For example, as illustrated in FIG. 13, the inner core member 2100a can define a corner 2180 at the transition from the portion of the inner core member 2100a that is disposed within first intermediate outer body member 2070a to the portion of the inner core member 2100a that defines first intermediate radiopaque portion 2064a of thread 2050a. As another example, as illustrated in FIG. 14, the inner core member 2100b can define a chamfer 2190 at the transition from the portion of the inner core member 2100b that is disposed within first intermediate outer body member 2070b to the portion of the inner core member 2100b that defines first intermediate radiopaque portion 2064b of thread 2050b.

In all embodiments, the inner core member can be made of any material suitable for use in medical devices intended for orthopedic use, including use as a long-term implant. Examples of suitable materials include metals, metal alloys, and polymeric materials. Examples of suitable metals include, but are not limited to, Titanium, Magnesium, and other metals. Examples of suitable metal alloys include, but are not limited to, Ti6Al4V, 316 LVM, 1.4441Ti-13Nb-13Zr, Ti-12Mo-6Zr-2Fe, Ti-15Mo-5Zr-3Al, Ti-15Mo, Ti-35Nb-7Zr-5Ta and Ti-29Nb-13Ta-4.6Zr Ti-6Al-7Nb and Ti-15Sn-4Nb-2Ta-0.2Pd Co—Cr—Mo alloys. Examples of suitable polymeric materials include, but are not limited to, polyaryletherketone (PAEK), polyether ether ketone (PEEK), PEEK (90G, 450G, I2, I4), Polyamid, PA66, carbon fiber reinforced polyaryletherketone (CFR PAEK), polyethere ketone ketone (PEKK), carbon fiber reinforced polyether ketone ketone (CFR PEKK), carbon fiber reinforced polyether ether ketone (CFR PEEK), CFR PEEK (90G CA30, 90G CA20, 450G CA30, 450G CA20, I2 CF20, I2 CF30, I4 CF30, I4 CF20), Polyamid CFR, and PA66 CFR.

In all embodiments, all outer body member components can be made of any material suitable for use in medical devices intended for orthopedic use, including use as a long-term implant. Examples of suitable types of materials include, but are not limited to, polymeric materials, blended materials such as carbon fiber reinforced polymers, and other materials. Examples of suitable polymeric materials include, but are not limited to, PAEK, CFR PAEK, PEKK, CFR PEKK, PEEK, CFR-PEEK, PEEK (90G, 450G, I2, I4), Polyamid, and PA66.

Examples of suitable blended materials include, but are not limited to, PEEK-Carbon materials, CFR PAEK, CFR PEKK, CFR PEEK (90G CA30, 90G CA20, 450G CA30, 450G CA20, I2 CF20, I2 CF30, I4 CF30, I4 CF20), Polyamid CFR, PA66 CFR.

It is noted that the materials used in a component of an orthopedic screw according to a particular embodiment can include additives, coatings, fillers, and/or other elements if desired. For example, antibiotics, bioactive glass, silver, copper, or another material that can reduce bacterial colonization of the orthopedic screw following implantation can be included in the material of the inner core member, the outer body member, or both. Furthermore, one or more components of an orthopedic screw according to an embodiment can be treated in a manner that facilitates making of the orthopedic screw, provides structural benefit to the orthopedic screw, or that provides other advantages. For example, in embodiments in which the inner core member comprises a metal, the inventors have determined that anodizing the inner core member in an orthopedic screw according to an embodiment prior to overmolding the inner core member with a suitable material to form the outer body member can be advantageous at least because anodization provides additional surface area on the inner core member to which the material of the outer core member can attach or bond during the overmolding process. Accordingly, an orthopedic screw according to any example described herein, or any other embodiment, can include a metal inner core member that comprises an anodized inner core member. In these embodiments, conventional anodization processes can be used to prepare the metal inner core member prior to overmolding the outer body member to form the orthopedic screw.

The inventors have determined that an orthopedic screw having an inner core member formed of a Titanium alloy, such as Ti6Al4V, and outer body member components formed of CFR PEEK provides desirable characteristics and a favorable balance between manufacturability and strength considerations.

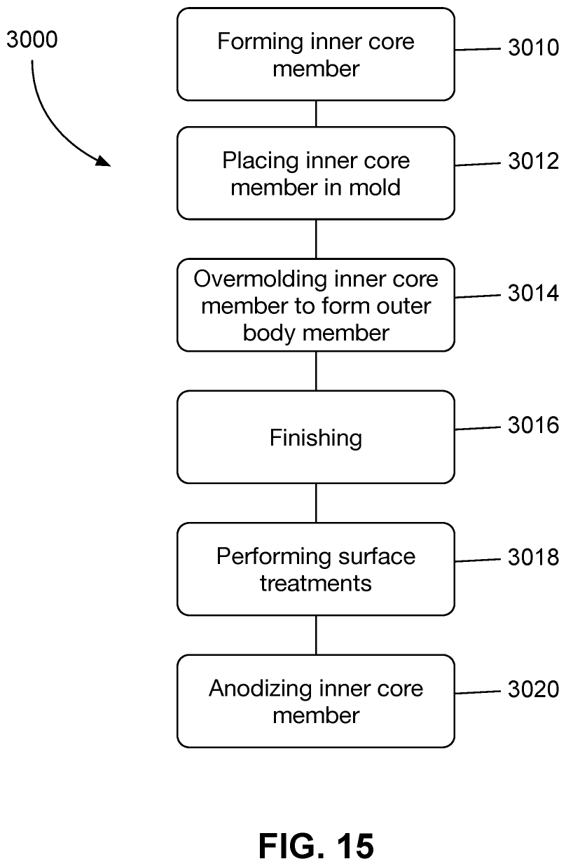

FIG. 15 is a schematic representation of a method 3000 of manufacturing an orthopedic screw. An initial step 3010 comprises forming an inner core member from a suitable precursor, such as a solid rod or cannula. This step 3010 is performed such that the inner core member defines a proximal radiopaque thread, a distal radiopaque thread, and a circumferential recess. A next step 3012 comprises placing the inner core member into a suitable mold. A next step 3014 comprises overmolding the inner core member with a suitable material to form an outer body member about the inner core member in accordance with an embodiment of the invention, including any of the examples described and illustrated herein. This step is performed such that the outer body member is disposed in the circumferential recess of the inner core member. Also, this step is performed such that the outer body member defines a radiolucent thread that is continuous with both the proximal radiopaque thread and the distal radiopaque thread. At this point, a medical device, such as an orthopedic screw, is available.

Step 3010 can be performed to such that the inner core member defines additionoal radiopaque threads, such as the intermediate radiopaque threads 2064, 2066 described above. Also, step 3014 can be performed such that a non-thread defining proximal outer body member is formed about the inner core member, such as proximal outer body member 2200 described above. Also, step 3014 can be performed such that additional outer body members are formed about the inner core member, such as intermediate outer body membes 2400, 2500 described above.

A method according to an embodiment can include various additional optional steps. For example, a step 3016 of finishing the orthopedic screw using suitable techniques or processes can be included. Also, a step 3018 of performing one or more surface treatments on the orthopedic screw, such as roughening, coating, and the like, can be included. Also, a step 3020 of anodizing the inner core member prior to performance of step 3014 of overmolding the inner core member with a suitable material to form the outer body member about the inner core member can be included. Inclusion of this step can be advantageous in methods of making an orthopedic screw in which it is desiable to increase the surface area on the inner core member to which the material of the outer core member can attach or bond during the step 3014 of overmolding the inner core member with a suitable material to form the outer body member.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular examples disclosed herein have been selected by the inventors simly to describe and illustrate examples of the invention are not intended to limit the scope of the invention or its protection, which is to be given the full breadth of the appended claims and any and all equivalents thereof.

We claim:

1. An orthopedic screw, comprising:
an inner core member defining a proximal radiopaque thread, a distal radiopaque thread, and a circumferential recess defining a circumferential recess floor; and
an outer body member disposed about the inner core member in the circumferential recess and defining a radiolucent outer body member external thread that is continuous with the proximal radiopaque thread and the distal radiopaque thread.

2. The orthopedic screw of claim 1, wherein the inner core member comprises an anodized metal.

3. The orthopedic screw of claim 2, wherein the outer body member comprises a polymeric material.

4. The orthopedic screw of claim 3, wherein the inner core member comprises a titanium alloy and the outer body member comprises CFR PEEK.

5. The orthopedic screw of claim 1, wherein the circumferential floor defines a plurality of circumferential grooves; and
wherein the outer body member extends into the pluarity of circumferential grooves.

6. The orthopedic screw of claim 1, wherein the circumferential floor defines a spiral groove; and
wherein the outer body member extends into the spiral groove.

7. The orthopedic screw of claim 1, wherein the circumferential floor defines a spiral projection.

8. The orthopedic screw of claim 1, wherein one of the proximal radiopaque thread and the distal radiopaque thread defines a reverse cutting tooth.

9. The orthopedic screw of claim 1, wherein the proximal radiopaque thread defines a reverse cutting tooth.

10. An orthopedic screw, comprising:
an inner core member defining a proximal radiopaque thread, a distal radiopaque thread, and an intermediate radiopaque thread disposed between and axially spaced from the proximal radiopaque thread and the distal radiopaque thread;
a first outer body member defining a first radiolucent outer body member external thread that is continuous with the proximal radiopaque thread and the intermediate radiopaque thread; and
a second outer body member defining a second radiolucent outer body member external thread that is continuous with the intermediate radiopaque thread and the distal radiopaque thread.

11. The orthopedic screw of claim 10, wherein the inner core member comprises an anodized metal.

12. The orthopedic screw of claim 11, wherein each of the first outer body member and the second outer body member comprises a polymeric material.

13. The orthopedic screw of claim 12, wherein the inner core member comprises a titanium alloy and each of the first outer body member and the second outer body member comprises CFR PEEK.

14. The orthopedic screw of claim 13, wherein one of the proximal radiopaque thread and the distal radiopaque thread defines a reverse cutting tooth.

15. The orthopedic screw of claim 13, wherein the proximal radiopaque thread defines a reverse cutting tooth.

16. An orthopedic screw, comprising:
an inner core member having a head having a first outer diameter, a tip having a second outer diameter, and defining a circumferential recess having a third outer diameter that is less than the first outer diameter and the second outer diameter, the inner core member defining a proximal radiopaque thread and a distal radiopaque thread; and
an outer body member disposed in the circumferential recess of the inner core member and defining a radiolucent outer body member external thread that is continuous with the proximal radiopaque thread and the distal radiopaque thread.

17. The orthopedic screw of claim 16, wherein the inner core member comprises an anodized metal.

18. The orthopedic screw of claim 17, wherein the outer body member comprises a polymeric material.

19. The orthopedic screw of claim 18, wherein the inner core member comprises a titanium alloy and the outer body member comprises CFR PEEK.

20. The orthopedic screw of claim 19, wherein one of the proximal radiopaque thread and the distal radiopaque thread defines a reverse cutting tooth.
